(19) United States
(12) Patent Application Publication
(10) Pub. No.: US 2026/0256976 A1
(43) Pub. Date: Sep. 3, 2026

(54) PHOTOCATALYTIC GATE ESPECIALLY FOR AIR AND SURFACE DISINFECTION

(71) Applicant: Piotr Czech, Trzebinia (PL)

(72) Inventor: Piotr Czech, Trzebinia (PL)

(21) Appl. No.: 18/866,090

(22) PCT Filed: Apr. 12, 2023
(86) PCT No.: PCT/IB2023/053731

§ 371 Date: Nov. 15, 2024

(30) Foreign Application Priority Data
May 16, 2022 (PL) P.441199

(51) Int. Cl. A61L 9/20; B01J 21/06; B01J 21/20; B01J 35/30; B01J 35/39; B01J 35/45; B01J 35/56; B01J 38/04

(52) U.S. Cl. CPC A61L 9/205; B01J 21/063; B01J 21/20; B01J 35/39; B01J 35/395; B01J 35/45; B01J 35/56; B01J 38/04; A61L 2202/11; A61L 2209/14; A61L 2209/212

(57) ABSTRACT

Photocatalytic gate, especially for air and surface disinfection, using a catalytic filter with a TiO₂ coating and a UVC lamp, characterized in that the device consists of: preferably alternately arranged in layers—rows parallel or perpendicularly or at an angle, at least one, preferably two rows of catalytic filters (FK), preferably finished with another third row in the form of an air filter (FP),
the catalytic filters (FK)-(FK1) and (FK2) are selected for the entire section of the air supply duct (K) or the section of the air handling unit;
between the first (FK1) and the second catalytic filter (FK2) there are placed UVC(L) lamps, preferably in the range of 185-253.7 nm, generating ozone O₃, which are used to excite the photocatalysis process on the coating (P) of the catalytic filter (FK) and generating ozone O₃,
and the whole structure is mounted—preferably on rails (S) made of stainless steel, fixed at the bottom and top of the duct (K), for easy removal and replacement of filters (FK1), (FK2) from the duct (K) or the air handling unit,
the gate is fully installed in the air supply duct (K) or the air handling unit of the ventilation, air-conditioning or forced-air heating system, in a place where the flowing air is already pre-cleaned of dust by air filters.

The subject of the invention is a photocatalytic gate, especially for air and surface disinfection.

Is well known photocatalysis technology for air and surface disinfection, which was developed by NASA scientists many years ago for space missions, in order to reduce Volatile Organic Compounds (VOCs) in the ventilation air.

All available devices on sale are devices with a low power UVC light source and a small area of the catalytic filter.

### Lamps Available in the World in Photocatalysis Technology:

1. PHT photocatalysis technology—photocatalysis technology,
2. RCI ActivePure® technology—Radial Catalytic Ionization
3. PCO™ technology—photocatalytic oxidation

From the publication “A Comparison of Photocatalytic Oxidation Reactor Performance for Spacecraft Cabin Trace Contaminant Control Applications Jay L. Perry1, Kenneth R. Frederick2, and Joseph P. Scott” the technology of photocatalysis—Photocatalytic oxidation (PCO) is known.

From the Chinese utility model description No. CN207262540U, an air purifying device based on light catalytic technology is known, including an induced air device, a filtering equipment, a purifier and an air removal device; the filter equipment includes a filter duct, a filter duct inlet and a threaded air outlet port of the air intake cylinder, be equipped with a filter duct, if the dry filtration mesh, the purifier contains a purifier pipe, be equipped with a plurality of purifier elements in the purifier pipe, contains a cylindrical shell for component purification, inside both ends of the coating are equipped with a catalyst plate, the last photocatalysis, which is equipped with a catalyst plate, be equipped with a light source between the two catalyst plates, be equipped with a light-reflecting coating on the inner wall of the coating, reflects the light that the reflective coating has given off light source for cover Talyst plate included, be equipped with replacement device on the purifier, air outlet device includes dryer outlet, dehumidifier and purifier tube threaded outlet, good purification effect, long service life is just convenient for change.

From the Chinese utility model description No. CN204768273U, photocatalysis waste gas purification system is known, collect the waste gas to cover and connect to the spray column, connect the glassed oxygen catalytic unit after the spray column, reconnect has the air induced device after the light oxygen catalytic unit, through the first filter to pass through, the cross-flow filter through the second between the light oxygen catalytic unit and the air induced device between the light oxygen catalytic unit and the spray column, continue to be equipped with a multi-channel tiO2 filter screen and UVC tube and light oxygen ozone generator catalytic unit, in which the multi-channel tiO2 filter and the interval of the UVC tube and the ozone generator are set. The utility model reveals that it combines the atomization technique of waste gas treatment with cooling, also can remove dust in partly waste gas beforehand, pre-treatment, and achieve a clear purpose, which keeps UVC manageable, at the same time removes the catalyst, under the influence of catalytic catalyst, greatly improve the efficiency photodissociative oxidation, the backside lowers the temperature, is washed beforehand into the complex exhaust gas of high temperature exhaust gas or composition, and the secondary light oxygen catalytic unit performs post-treatment, is to overcome the shortcoming of adoptive monotechnic.

US2014234163A1 discloses a method of air purification comprising the degradation of all or some pollutants by the action of UV rays, which is achieved with a single light source emitting rays with a wavelength of 254 nm and rays with a wavelength of 185 nm, and optionally UVA rays and by photocatalysis the ratio of the amount of energy 185 to the amount of energy 254 ranges from 0.01 to 1. The present invention also relates to a device enabling said method to be carried out.

From the Chinese patent description No. CN109603534A, an air sterilization device based on photocatalysis technology is known. The air sterilization device consists of a housing, a filter, a photocatalysis module, a draft fan, a power supply circuit board and a control circuit board, the air inlet is formed in the upper part of the housing, the air outlet is formed in the bottom of the shell, a hollow plug and a coarse filter mesh are placed on the air inlet, the coarse filter mesh and the hollow plug are compactly connected to the air inlet, and the filter, photocatalysis module, draft fan, power circuit board and control circuit board are placed in the shell; the filter is installed under the air inlet and consists of a filter element and a HEPA filter mesh, the filter element is located on top of the HEPA filter mesh, the photocatalysis module is located under the filter and consists of a photocatalytic mesh and excitation light sources, which together, the photocatalysis mesh is covered photocatalyst coating, and the excitation light sources are placed between the photocatalysis grid. With the air sterilization device based on photocatalysis technology, organic pollutants such as formaldehyde, benzene, methylbenzene, xylene, olefin, aromatic hydrocarbon and TVOC gas can be removed, air quality is improved and human health is protected

The aim of the invention is to develop a new construction—a photocatalytic gate for effective—effective disinfection of air and surfaces, which through the appropriate construction allows—to increase the surface of the photocatalyst and artificially generate ozone used simultaneously with UVC and/or UVA irradiation of the active coating (P) of the catalytic filter.

The gate according to the invention is characterized in that the device consists of at least one, preferably two, rows of catalytic filters (FK) arranged alternately in layers—rows parallel or perpendicularly or at an angle, preferably ended with another third row in the form of an air filter (FP),

the catalytic filters (FK)-(FK1) and (FK2) are selected for the entire section of the air supply duct (K) or the section of the air handling unit;
between the first (FK1) and the second catalytic filter (FK2) there are placed UVC(L) lamps, preferably in the range of 185-253.7 nm, generating ozone O₃, which are used to excite the photocatalysis process on the coating (P) of the catalytic filter (FK) and generating ozone O₃,
and the whole structure is mounted—preferably on rails (S) made of stainless steel, fixed at the bottom and top of the duct (K), for easy removal and replacement of filters (FK1), (FK2) from the duct (K) or the air handling unit, whereby the whole gate is mounted in the air supply duct (K) or the air handling unit of the ventilation, air conditioning or blow heating system, in a place where the flowing air is already pre-cleaned of dust by air filters.
FK1), a filter made of perforated stainless steel, preferably chromium-nickel and/or copper, with dimensions of mesh size 2-4 mm and thickness 0.50 mm.
As the second catalytic filter (FK2), a filter made of perforated stainless steel, preferably chromium-nickel and/or copper, with dimensions of mesh diameter 2-4 mm and thickness 0.50 mm.
The catalytic filters (FK1) and (FK2) are covered with a coating (P) of nanotitanium dioxide-Tio₂ with a thickness of at least 20 nm, preferably 30-40 m.
The (P) coating of nanotitanium dioxide—Tio₂ is hot-dip and/or spray-coated on a chromium-nickel and/or copper metal grid, while the adhesion of TiO₂ to the substrate is preferably obtained by plasma treatment of the pre-prepared layer.
The coating (P) has admixtures in the form of copper, nanocopper and/or nanosilver, preferably up to 25% of the weight of the entire applied coating (P).
P) has additional layers and/or stripes in the form of copper, nanocopper and/or nanosilver applied, preferably up to 50% of the weight of the total applied coating (P).
Instead of UVC lamps (L), the following are used: LED lamps, UVC and/or UVA LED fluorescent lamps/strips, UVA lamps, lamps or devices generating UVC.
For every 1000 m³/h of ventilation air flow in the duct (K), it is preferable to use from 0.25 to 0.3 m2 of the surface of the catalytic filter (FK); and at the same time the surface of the catalytic filter (FK1) and (FK2) covers the entire cross-section of the ventilation duct (K) or the unit.
Inside the gate, i.e. on the first catalytic filter (FK1) and on the second catalytic filter (FK2), a flow/atmosphere of at least 10 μg/m³ ozone, preferably 40 to 150 μg/m³—of ozone is generated for at least 1 minute, preferably 1 hour; with simultaneous illumination of the coating surface (P) of the catalytic filters (FK1) and (FK2) by UVC and/or UVA radiation.
The construction of the gate device allows for any expandable configuration of the active surface of the catalyst with nano Tio₂—coatings (P)—scalability, so that there is no limitation as to the use of the surface, and thus obtaining the desired biocidal effectiveness.
The construction of the gate device allows, using mounting rails (S), to conveniently and quickly slide out the catalyst cassette for its regeneration—application of a nano Tio_{2 coating}.
Regeneration of the TiO_{2 catalytic filter} is carried out by spraying the agent with a battery atomizer or by manually applying the coating (P).
UVC or UVA (L) LED fluorescent lamps/strips.

The essence of the operation of the gate according to the invention is the use of a double catalytic filter with a UVC lamp generating ozone O (at least one lamp) or an ozone-free UVC lamp and an ozone generator O₃ in the middle.

The air passes through FK1+UVC, where hydroxyl radicals OH— and hydrogen peroxide H₂O₂ are formed, then they combine with O₃ and most likely hydroperoxide radicals (HO₂^{o}) and anion superoxide radicals (O₂^{o-}) are formed, then the air passes through FK2+UVC and further combinations of compounds are created that effectively eliminate pollution—they disinfect the air and surfaces.

The photocatalytic gate according to the invention uses the photocatalysis process consisting in the use of a nano-titanium dioxide (TiO₂) coating, which under the influence of UVC radiation acts as a semiconductor, causing the transfer of electrons from the valence layer to the conduction layer, thanks to which very active compounds are formed, such as: hydrogen peroxide (H₂O₂) and hydroxyl radicals (OH).

One of the key elements of the solution is the use of a large area of photocatalytic filters (the area of which is 10 to 20 times larger than the existing solutions) and UVC lamps in the range of 185-253.7 nm generating small amounts of ozone O₃, thanks to which hydroperoxide radicals are most likely formed (HO 2°) and superoxide anion radicals (O₂^{o-}).
Due to the very strong oxidizing effect, they effectively decompose more complex compounds into simpler forms with a slight emission of carbon dioxide and the formation of water molecules.
Increasing the surface area of the photocatalyst and artificial generation of O 3 will allow for a wider application of the technology in the pharmaceutical, food and agricultural industries (hatcheries, farms, cowsheds, piggeries).
So far, the available lamps in photocatalysis technology with a limited catalyst surface and the lack of artificially generated O ₃ have not been able to ensure adequate efficiency and thus biocidal effectiveness at large volumes of objects or high microbial load.
Any attempts to increase the efficiency were associated with the need to use a large number of lamps, which increased the installation costs to economically unacceptable by investors. In addition, the photocatalytic coating used was unstable, of poor quality and had a very small active-working surface.
The air filter (FK) is used to protect the filters (FK1) and (FK2) against dust—when it is installed as the third one in relation to the air flow in the duct (K), it catches the detached particles of the TiO₂ coating.

### The Gate According to the Invention Works in Two Stages:

1. Disinfects the flowing air, thanks to the appropriate UVC light power
at the level of 90 to 99.9%;
2. Disinfects ventilation ducts as well as indoor air and surfaces,
to which the ventilation air reaches, thanks to the use of the Tio₂ catalytic filter with a large surface area that generates large amounts of hydroxyl (OH—).

### The Advantages of the Construction According to the Invention are:

Reducing the spread of bacteria and viruses among humans and animals (including H1N1/flu, coronavirus-229E/cold, SARS-CoV-2/covid19, Staphylococcus aureus/Staphylococcus aureus, Salmonella Enterica, Enterococcus faecalis/Fecal streptococcus)
Reduction of infections among the staff of the Health Service
Minimization of nosocomial infections
Reducing the number of absenteeism due to illness among employees, pre-school and school children and students
Perhaps the photocatalytic gate according to the invention is effective against ASF (African Swine Fever) and Avian flu, —additional studies and tests must be carried out to confirm the effectiveness with a specific catalyst surface—coating (P) of the catalytic filter irradiated with UVC and/or UVA and the proposed high concentration of ozone, used simultaneously with irradiation.

### Additional Advantages of the Structure According to the Invention are:

The design of the device allows for any configuration of the surface of the Nano TiO₂ catalyst (there is no limitation as to the use of the surface) and thus obtaining the appropriate biocidal effectiveness;
The design of the device allows for convenient and quick removal of the catalyst cassette for its regeneration (coating with Nano TiO₂);
Regeneration of the TiO₂ catalytic filter is carried out by spraying the agent with a battery atomizer or by hand with a brush/roller;
Easy service access to UVC or UVA LED tubes/strips.

The subject of the invention will be used in limiting the spread of bacteria and viruses through effective disinfection of air and surfaces in medical facilities, public facilities, laboratories, hotels, pharmaceutical and food industries, educational facilities, in livestock buildings (farms, cowsheds, piggeries, etc.)

The subject of the invention is explained in the examples of embodiment in the drawings in which:

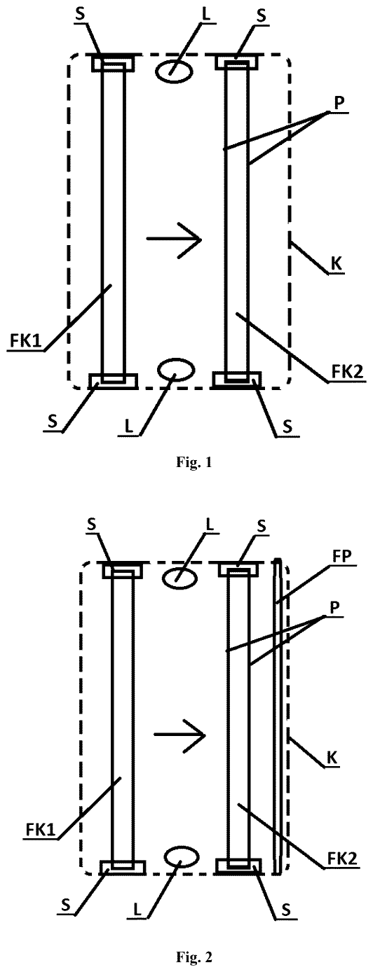

FIG. 1 shows the gate according to the invention—two rows of catalytic filters—model on the left, practical implementation on the right;

FIG. 2 shows the gate according to the invention—two rows of catalytic filters with a third row of air filters;

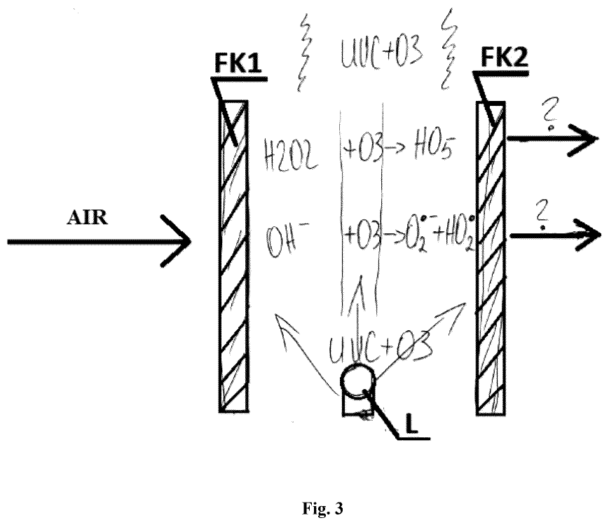

FIG. 3 shows a diagram of the photocatalytic gate Tio₂+UVC+O ₃, where the resulting compounds are marked.

### EXAMPLE 1 (2 ROWS, NO AIR FILTER)

The device consists of two rows of catalytic filters (FK) placed alternately in parallel rows in layers.

Catalytic filters (FK)-(FK1) and (FK2) are selected for the entire cross-section of the supply duct (K).

Between the first (FK1) and the second catalytic filter (FK2) there are UVC(L) lamps, in the range of 185-253.7 nm, generating ozone O₃, which are used to excite the photocatalysis process on the coating (P) of the catalytic filter (FK) and generating ozone O₃,

The entire structure is mounted on rails (S) made of stainless steel, fixed at the bottom and top of the duct (K), for easy removal and replacement of filters (FK1) and (FK2) from the duct (K).

The gate is fully installed in the air supply duct (K) or the air handling unit of the ventilation, air conditioning or blow heating system, in a place where the flowing air is already pre-cleaned of dust by air filters.

As the first catalytic filter (FK1), a filter made of perforated stainless chromium-nickel sheet with dimensions of mesh size 2-4 mm and thickness 0.50 mm.

As the second catalytic filter (FK2), a filter made of perforated stainless chromium-nickel and copper sheet with dimensions of mesh diameter 2-4 mm and thickness 0.50 mm.

Catalytic filters (FK1) and (FK2) are covered with a coating (P) of nano-titanium dioxide—Tio₂ with a thickness of 30-40 μm.

Coating (P) of nanotitanium dioxide—Tio₂ is spray-coated on a metal mesh, while the adhesion of TiO₂ to the substrate is enhanced by plasma treatment of the pre-prepared layer.

The coating (P) has admixtures in the form of copper 10% of the weight of the entire applied coating (P).

The coating (P) has additional layers and/or stripes of nanosilver applied (up to 10% of the weight of the entire applied coating (P)).

Instead of UVC (L) lamps, the following are used: LED lamps, UVC and/or UVA LED fluorescent lamps/strips, UVA lamps, lamps or devices generating UVC environment.

For every 1000 m³/h of ventilation air flow in the duct (K), it is good to use from 0.25 to 0.3 m² of the surface of the catalytic filter (FK); and at the same time the surface of the catalytic filter (FK1) and (FK2) covers the entire cross-section of the ventilation duct (K) or the unit.

Inside the gate, ie on the first catalytic filter (FK1) and on the second catalytic filter (FK2) a flow/atmosphere of 50 μg/m, ozone 1 hour is generated; with simultaneous illumination of the coating surface (P) of the catalytic filters (FK1) and (FK2) by UVC and/or UVA radiation. The construction of the gate device allows for any expandable configuration of the active surface of the catalyst with nano Tio₂—coatings (P)—scalability, so that there is no limitation as to the use of the surface, and thus obtaining the desired biocidal effectiveness.

The construction of the gate device allows, using mounting rails (S), to conveniently and quickly slide out the catalyst cassette for its regeneration—application of a nano Tio₂ coating. Regeneration of the TiO₂ catalytic filter is carried out by spraying the agent with a battery atomizer or by manually applying the coating (P).

The design allows easy service access to UVC or UVA (L) LED fluorescent lamps/strips.

### EXAMPLE 2 (3 ROWS, WITH AIR FILTER)

The device consists of two rows of catalytic filters (FK) arranged alternately in parallel rows in layers, ended with another third row in the form of an air filter (FP),

Catalytic filters (FK)-(FK1) and (FK2) are selected for the entire section of the air supply duct (K) or the section of the air handling unit;

Between the first (FK1) and the second catalytic filter (FK2) there are UVC(L) lamps, in the range of 185-253.7 nm, generating ozone O₃, which are used to excite the photocatalysis process on the coating (P) of the catalytic filter (FK) and generating ozone O₃,

The entire structure is mounted on rails (S) made of stainless steel, fixed at the bottom and top of the duct (K), for easy removal and replacement of filters (FK1), (FK2) from the duct (K) or the air handling unit.

The gate is fully installed in the air supply duct (K) or the air handling unit of the ventilation, air conditioning or blow heating system, in a place where the flowing air is already pre-cleaned of dust by air filters.

As the first catalytic filter (FK1), a filter made of perforated stainless chromium-nickel sheet with dimensions of mesh size 2-4 mm and thickness 0.50 mm.

As the second catalytic filter (FK2), a filter made of perforated stainless chromium-nickel and copper sheet with dimensions of mesh size 2-4 mm and thickness 0.50 mm.

Catalytic filters (FK1) and (FK2) are covered with a coating (P) of nano-titanium dioxide—Tio₂ with a thickness of 40 μm.

The (P) coating of nanotitanium dioxide—Tio₂ is spray-coated on a chromium-nickel and copper metal grid, while the adhesion of TiO₂ to the substrate is enhanced by plasma treatment of the pre-prepared layer.

The coating (P) has admixtures in the form of nanocopper 15% of the weight of the entire applied coating (P).

The coating (P) has additional layers and/or stripes in the form of nanosilver applied, 5% of the weight of the entire applied coating (P).

Instead of UVC lamps (L), the following are used: LED lamps, UVC and/or UVA LED fluorescent lamps/strips, UVA lamps, lamps or devices generating UVC.

For every 1000 m³/h of ventilation air flow in the duct (K), it is good to use from 0.25 to 0.3 m² of the surface of the catalytic filter (FK); and at the same time the surface of the catalytic filter (FK1) and (FK2) covers the entire cross-section of the ventilation duct (K) or the unit.

Inside the gate, ie on the first catalytic filter (FK1) and on the second catalytic filter (FK2) a flow/atmosphere of 100 μg/m³ ozone is generated for 30 minutes; with simultaneous illumination of the coating surface (P) of the catalytic filters (FK1) and (FK2) by UVC and/or UVA radiation.

The design of the gate device allows for any expandable configuration of the active surface of the catalyst with nano Tio₂—coatings (P)—scalability, so that there is no limit to the use of the surface, and thus obtaining the desired biocidal effectiveness.

The construction of the gate device allows, using mounting rails (S), to conveniently and quickly slide out the catalyst cassette for its regeneration—application of a nano Tio₂ coating.

Regeneration of the TiO₂ catalytic filter is carried out by spraying the agent with a battery atomizer or by manually applying the coating (P).

The design allows easy service access to UVC or UVA (L) LED fluorescent lamps/strips.

Air filter (FP)—prevents clogging of catalytic meshes by dust and dust.

### EXAMPLE 3 (HATCHERY AND POULTRY FARM IMPLEMENTATION)

TLV for O₃ is 150 μg/m³.

Measurements made on the object were made three times: (ozone values, maintained ozone atmosphere)
Sorting plant—151 μg/m³ (people stay 2×8 hours a week)
Chick Warehouse 2-192 μg/m³
Chick Warehouse 1-194 μg/m³
Corridor hatchers—49 μg/m³
Egg yield (ventilation step 1 out of 5 steps)—40 μg/m³
Egg storage—51 μg/m³
Washing of hatching carts and trays—51 μg/m³ (people stay 1×2-4 hours during the week)

In connection with the above, it was found that the necessary concentration of O₃ on the object for the proper course of the photocatalysis+O₃ process ranges from 40 μg/m³ to 150 μg/m³.

### As a Result of the Laboratory Test, the Bactericidal Effectiveness was Found:

1.2 Test for Photocatalysis Alone (without Ozone) on Farm:

Microbiological parameters—testing the bactericidal effectiveness against
1. Enterococcus faecolis ATCC 29212
2 Salmonella enterica subsp. Enterica serovar Enteritidis ATCC 13076

The degree of reduction of microorganisms increased with the extension of the exposure of bacteria to the device and was after 2 hours for:
ad1 −15.92% and
ad2. −31.34%.

### 2. Proper Examination—Solution According to the Invention:

Samples surface swabs on a sponge taken from: sorting plant, hatching halls, warehouses, washing of hatcher baskets, brooders, hatching halls surface swabs on a sponge taken from the stock/swimming pool+egg warehouse—surface swabs on a sponge taken from the social room
Collection period: after disinfection
Sampling site: Hatchery and Poultry Farm
Presence and identification of Salmonella.
Breeding method with biochemical and serological confirmation.

### PN-EN ISO 6579-1:2017-04

White-Kaufmann-Le Minor scheme. 2007 (A)
conclusion of the test: after disinfection—Salmonella not detected in the analytical sample

In our solution according to the invention, after 2-3 hours, preferably 24 hours, 100% disinfection of salmonella takes place.

### List of Elements

FK—catalytic filters
FK1—the first catalytic filter
FK2—second catalytic filter
FP—air filter
UVC lamps
P—coating
K—air supply duct
S—mounting rails Glossary of terms:
PHT photocatalysis technology—photocatalysis technology,
PHT Photon Hydroxylation Technology—photohydroxylation technology
RCI ActivePure® Technology—Radial Catalytic Ionization
PCO™ technology—photocatalytic oxidation
NDS—the value of the maximum allowable concentrations

1. Photocatalytic gate, especially for air and surface disinfection, using a catalytic filter with a TiO₂ coating and a UVC lamp, characterized in that the device consists of: preferably alternately arranged in layers—rows parallel or perpendicularly or at an angle, at least one, preferably two rows of catalytic filters (FK), preferably finished with another third row in the form of an air filter (FP),
the catalytic filters (FK)-(FK1) and (FK2) are selected for the entire section of the air supply duct (K) or the section of the air handling unit;
between the first (FK1) and the second catalytic filter (FK2) there are placed UVC(L) lamps, preferably in the range of 185-253.7 nm, generating ozone O₃, which are used to excite the photocatalysis process on the coating (P) of the catalytic filter (FK) and generating ozone O₃,
and the whole structure is mounted—preferably on rails (S) made of stainless steel, fixed at the bottom and top of the duct (K), for easy removal and replacement of filters (FK1), (FK2) from the duct (K) or the air handling unit,
the gate is fully installed in the air supply duct (K) or the air handling unit of the ventilation, air-conditioning or forced-air heating system, in a place where the flowing air is already pre-cleaned of dust by air filters.

2. The gate according to claim 1, characterized in that the first catalytic filter (FK1) is made of perforated stainless steel, preferably chromium-nickel and/or copper, with dimensions mesh size 2-4 mm and thickness 0.50 mm.

3. The gate according to claim 1, characterized in that the second catalytic filter (FK2) is made of perforated stainless steel, preferably chromium-nickel and/or copper, with dimensions mesh diameter 2-4 mm and thickness 0.50 mm.

4. The gate according to claims 1, 2, 3, characterized in that the catalytic filters (FK1) and (FK2) are covered with a coating (P) of nanotitanium dioxide—Tio₂ with a thickness of at least 20 nm, preferably 30-40 m.

5. The gate according to claims 1 and 4, characterized in that the coating (P) of nanotitanium dioxide—Tio₂ is hot-dip and/or spray-coated on a chromium-nickel and/or copper metal grid, with the reinforcement of TiO₂ adhesion to the substrate being advantageously obtained as a result of plasmaizing the pre-prepared layer.

6. The gate according to claims 1 and 4, characterized in that the coating (P) has admixtures in the form of copper, nanocopper and/or nanosilver, preferably up to 25% of the weight of the entire applied coating (P).

7. The gate according to claims 1 and 4, characterized in that the coating (P) has additional layers and/or strips in the form of copper, nanocopper and/or nanosilver applied, preferably up to 50% of the weight of the entire applied coating (P).

8. The gate according to claim 1, characterized in that instead of UVC lamps (L), the following are used:
LED lamps, UVC and/or UVA LED fluorescent lamps/strips, UVA lamps, lamps or devices generating UVC environment.

9. The gate according to claim 1, characterized in that for every 1000 m³/h of ventilation air flow in the duct (K), it is preferable to use from 0.25 to 0.3 m² of the surface of the catalytic filter (FK); and at the same time the surface of the catalytic filter (FK1) and (FK2) covers the entire cross-section of the ventilation duct (K) or the unit.

10. The gate according to claim 1, characterized in that inside the gate, i.e. on the first catalytic filter (FK1) and on the second catalytic filter (FK2), a flow/atmosphere of at least 10 μg/m³ of ozone is generated, preferably 40 to 150 μg/m³, ozone for at least 1 minute, preferably 1 hour; with simultaneous illumination of the coating surface (P) of the catalytic filters (FK1) and (FK2) by UVC and/or UVA radiation.

11. The gate according to claim 1, characterized in that the construction of the gate device allows any expandable configuration of the active surface of the nano Tio₂ catalyst—shells (P)—scalability, so that there is no limitation as to the use of the surface, and thus obtaining the appropriate desired biocidal effectiveness.

12. The gate according to claim 1, characterized in that the structure of the gate device allows, by means of mounting rails (S), to conveniently and quickly slide out the catalyst cassette for its regeneration—application of the Tio₂ nano coating.

13. The gate according to claim 1, characterized in that regeneration of the TiO₂ catalytic filter is carried out by spraying the agent with a battery atomizer or by manually applying the coating (P).

14. The gate according to claim 1, characterized in that the structure allows easy service access to UVC or UVA LED fluorescent lamps/strips (L).
